SEQUENCE LISTING (14 sequences)

SEQ ID NO: 1 - DNA, 20 bp, Artificial
  cyp2d6 and cyp2d8 forward primer
cagggactgcgggagaccag

SEQ ID NO: 2 - DNA, 23 bp, Artificial
  CYP2D6 and CYP2D8 reverse primer
ccttcccagttcccgctttgtgc

SEQ ID NO: 3 - DNA, 20 bp, Artificial
  CYP2D6 reverse primer
ccatagcgcgccaggaacac

SEQ ID NO: 4 - DNA, 22 bp, Artificial
  CYP2D8 reverse primer
gtccgtagtgtgccagaaacac

SEQ ID NO: 5 - DNA, 33 bp, Artificial
  CYP2D6 amplicon discriminator
ctgagtccgaacattgagtttgcattagcccca

SEQ ID NO: 6 - DNA, 28 bp, Artificial
  CYP2D8 amplicon discriminator probe
gcagtatatcgcttgacacttggccccg

SEQ ID NO: 7 - DNA, 31 bp, Artificial
  CYP2D6 stabilizer
ccatccaccacccactccaaccctatgctcc

SEQ ID NO: 8 - DNA, 31 bp, Artificial
  CYP2D8 stabilizer
ccctccgccgcccactccaaccctgtgcttt

SEQ ID NO: 9 - DNA, 18 bp, Artificial
  green probe for 2D6.disc
ctcaatgttcggactcag

SEQ ID NO: 10 - DNA, 18 bp, Artificial
  red probe for 2D8.disc
tgtcaagcgatatactgc

SEQ ID NO: 11 - DNA, 12 bp, Artificial
  CYP2D6 probe
gcattagcccca

SEQ ID NO: 12 - DNA, 9 bp, Artificial
  CYP2D8 probe
ttggccccg

SEQ ID NO: 13 - DNA, 37 bp, Artificial
  CYP2D6 real-time beacon probe
tgggacggttcatggccacgcgcacgtgcccgtccca

SEQ ID NO: 14 - DNA, 37 bp, Artificial
  CYP2D6 pseudogene 8 beacon probe
cgggacggtccatgaccacgtccatgtgtccgtcccg

(19) United States
(12) Patent Application Publication
(10) Pub. No.: US 2009/0087846 A1
(43) Pub. Date: Apr. 2, 2009

(54) Method for Detecting Large Mutations and Duplications Using Control Amplification Comparisons to Paralogous Genes

(76) Inventors: Ray R. Radtkey, San Diego, CA (US); Douglas Whitman, Austin, TX (US); Graham Lidgard, La Jolla, CA (US)

Correspondence Address: MINTZ, LEVIN, COHN, FERRIS, GLOVSKY AND POPEO, P.C, 5 Palo Alto Square - 6th Floor, 3000 El Camino Real, PALO ALTO, CA, 94306-2155, US

(21) Appl. No.: 12/118,312

(22) Filed: May 9, 2008

Related U.S. Application Data
(63) Continuation of application No. 11/485,167, filed on Jul. 12, 2006, now abandoned.
(60) Provisional application No. 60/698,807, filed on Jul. 12, 2005.

(51) Int. Cl. C12Q 1/68

(52) U.S. Cl. 435/6

(57) ABSTRACT

Methods for querying biological samples to detect genetic mutations, particularly insertions and deletions, by co-amplification of a gene of interest in conjunction with a paralogous gene. When the gene of interest and the corresponding paralogous gene are selected from the CYP450 family, the resulting ratios may predict how a particular patient metabolizes certain prescription drugs.

### RELATED APPLICATION INFORMATION

This application is a continuation of U.S. application Ser. No. 11/485,167, filed Jul. 12, 2006, entitled “Method for Detecting Large Mutations and Duplications Using Control Amplification Comparisons to Paralogous Genes”, which claims priority to U.S. Provisional Patent Application Ser. No. 60/698,807, filed Jul. 12, 2005, which applications are hereby incorporated in their entirety.

### FIELD OF THE INVENTION

The methods of these inventions relate to detecting biological samples for genetic mutations, particularly insertions and deletions, by co-amplification of a gene of interest in conjunction with a paralogous gene. In particular, the invention enables both high throughput screening and identification of mutations in a biological sample. More particularly, the invention relates to methods for detecting mutations in the cytochrome P450 gene family, which methods may be useful in predicting how a patient metabolizes certain drugs.

### BACKGROUND OF THE INVENTION

Proteins encoded by the cytochrome P450 (CYP) gene family are involved in the degradation of many toxic and potentially toxic compounds. Of the more than 60 CYP genes known in the human genome, 6 are considered the most metabolically active. Many prescription drugs serve as substrates for the enzymes encoded by the CYP gene family. Some classes of prescription drugs that serve as substrates for enzymes encoded by the CYP family include tricyclic antidepressants, selective serotonin uptake inhibitors, antipsychotics, neuroleptics, anticancer agents, alpha-blockers, antiarrhythmics and opiates.

The CYP2D6 gene locus, for example, is a complex locus resulting from serial duplication events that rendered two highly homologous sequences, CYP2D7 and CYP2D8, located upstream. It is well known that point mutations, insertions and deletions, in the CYP gene family can lead to clinically important poor metabolizer (“PM”) and extensive metabolizer (“EM”) phenotypes that can affect significantly the outcome of drug therapy. The high levels of variability within the EM phenotype have led to classification of the most rapidly metabolizing phenotype as ultra-rapid metabolizers (“UM”) and the slowest of the extensive metabolizers as intermediate metabolizers (“IM”). The PM phenotype frequently comprises toxicity due to accumulation of active compounds and the subsequent lack of drug response resulting from the inability to activate the prodrug. Patients possessing the PM phenotype may develop toxic plasma concentrations of a prescribed drug potentially leading to exaggerated responses and other adverse reactions due to impaired drug metabolism even when administered a standard dose of treatment. The UM phenotype arises from duplication alleles inherited in a dominant fashion resulting in increased drug dosage and consequent under-dosing of drugs. Patients with the UM phenotype may suffer from therapeutic failure as a result of rapid drug metabolism.

The protein encoded by the CYP2D6 gene, debrisoquine 4-hydroxylase, is one of the more important enzymes linked to prescription drug metabolism. In fact, the protein is involved in degradation of approximately 100 prescription drug compounds. Approximately 10% of Caucasian population have reduced debrisoquine 4-hydroxylase activity levels resulting in decreased clinical response to a variety of prescription drugs. Conversely, approximately 7% of the population have increased levels of the enzyme, resulting in increased clinical response to the same variety of prescription drugs. Four CYP2D6 SNPs (*3, *4, *6 and *7), as well as a whole-gene deletion allele (*5), contribute to about 98% of the PM phenotype, while duplication alleles constitute all of the UM alleles.

The relationship between CYP2D6 gene copy number and phenotype has been well characterized. The deletion of the entire CYP2D6 gene, along with the functionality, was first shown by Gaedigk, et. al in 1991 (“Deletion of the entire cytochrome P450 gene is a cause of impaired drug metabolism in poor metabolizers of the debrisoquine/sparteine polymorphism.” Am. J Human Genetics, 48:943-950). Johansson, et. al in 1993 (“Inherited amplification of an active gene in the cytochrome P450 CYP2D locus as a cause of ultrarapid metabolism of debrisoquine.” Proc. Nat. Acad. Sci. USA 90:11825-11829) established the relationship in the context of the duplication of the CYP2D6 gene. In this case, individuals possessing extra functional gene copies (3 or greater) have faster metabolism of debrisoquine (lower metabolic ratio) than individuals with 2, 1 or 0 functional copies. Lovlie, et. al in 1997 (“Characterization of the 16+9 kb and 30+9 kb CYP2D6 XbaI haplotypes.” Pharmacogenetics 7:149-152) showed that the type of allele duplicated had to be taken into account. Those authors demonstrated the existence of the duplicated CYP2D6*4 allele. (Id.) The CYP2D6*4 allele is known to be non-functional (Kagimoto, et. al (1990) “Multiple mutations of the human cytochrome P45011D6 gene (CYP2D6) in poor metabolizers of debrisoquine. Study of the functional significance of individual mutations bu expression of chimeric genes.” J. Biol. Chemistry 265:17209-17214); thus having an extra copy of the non-functional allele does not lead to increased metabolism.

Given the important role of the CYP2D6 gene in prescription drug metabolism and the clinical impact of PM and UM phenotypes, the CYP2D6 gene has been one of the most popular targets for genetic analysis in order to determine a patient's individual drug therapy strategy. One beneficial example of a screen for determining a patient's CYP2D6 copy number is predicting a patients likely reaction to administration of the drug ondansetron, which is frequently administered to patients to mitigate post-operative nausea and vomiting. (Candiotti, et al., (2005) “The impact of pharmacogenetics on postoperative nausea and vomiting (PON and POV)—Do CYP2D6 copy number and polymorphisms affect the success of ondansetron prophylaxis?” Anesthesiology 102: 543-549). Candiotti, et al. concluded that ultra-rapid metabolizers have an increased chance of ondansetron failure to prevent POV and therefore frequently require an extra night in the hospital. (Id.)

The general implementation of pharmacogenetic testing will require accurate, efficient and flexible platforms. Developing such clinical assays for the CYP2D6 gene, however, involves several technical challenges. One challenge is that the CYP2D6 gene exists in the genome amid highly homologous pseudogenes. The existence of these pseudogenes complicates the specific amplification of the CYP2D6 gene product. Traditional analysis of the copy number of the CYP2D6 gene typically involves a strategy employing two rounds of nested PCR in order to avoid co-amplification of pseudogenes.

A second challenge is the high degree of CYP2D6 gene polymorphism, which includes the deletion (*5, *13 and *16 allele) or duplication (e.g., *1×N, *2×N, *4×N, *10×N, *35×N and *36×N alleles) of a whole active CYP2D6 gene, as well as many single-nucleotide polymorphisms (SNPs). Although numerous techniques are available for SNP detection, only 2 methods, PCR-RFLP (restriction fragment length polymorphism) and Long-Range PCR, are available for clinical detection of gene deletion and insertion. Both methods require special PCR reagents, expensive and rare DNA polymerases, extended amplification times, and a labor-intensive agarose gel analysis of the amplified products. For example, the market-leading CYP2D6 clinical analysis offered by Genaissance Pharmaceuticals (New Haven, Conn., USA), entails four separate assays, including Long-Range PCR, in order to identify CYP2D6 gene mutations. The Genaissance analysis has a standard turn-around time of 5 business days.

Although the Long-Range PCR method has been widely used for analysis of CYP2D6 gene deletion (*5 allele) and duplication, the method has several disadvantages. First, the Long-Range PCR method frequently fails to detect the *5 allele thereby yielding false negatives. Second, these methods may fail to detect other deletion alleles (*13 and *16) that have been generated by gene conversion. Third, Long-Range PCR relies on a relatively uncommon PCR reagent and requires a longer PCR reaction time which is incompatible with more traditional PCR analyses. Finally, the Long-Range PCR method relies on an agarose gel analysis of the PCR products which is difficult, if not impossible, to scale up for high throughput analysis and automation.

Although the basic principle underlying the present method is not new, all methods from previous studies use different primers for the test and internal control regions in the multiplex reaction. Due to a different annealing affinity and amplification efficiency by the different primers, the end-product ratio in such a reaction may not accurately represent the gene ratio existing in the genome. Therefore, in those reactions, it is essential to adjust the PCR conditions, including the concentrations for each primer, to accommodate a consistent and parallel linear amplification from both the test and control genes. To fulfill such task sometimes can be very challenging.

Accordingly, it would be desirable to achieve rapid and efficient co-amplification of a genetic locus of interest and a control genetic locus in a manner which overcomes the challenges noted above.

### SUMMARY OF THE INVENTION

The present invention provides methods for detecting mutations in genetic loci from a biological sample. The method contemplates isolation of genomic DNA from the biological sample, amplification of a portion of the gene of interest together with co-amplification of a control gene to produce target amplicons and control amplicons, respectively. The target and control amplicons are hybridized to probes bound to a support situated at predetermined locations. The amount of target and control amplicons are detected and their relative amounts are compared. A ratio is determined between the relative amounts of target and control amplicons that are hybridized, such that the ratio is indicative of the presence or absence of a genetic mutation in the gene of interest.

In one embodiment, a ratio of about 0:1 target amplicon to control amplicon indicates that the sample was taken from a patient who is homozygous for a deletion at the gene of interest. A ratio of about 0.5:1 target amplicon to control amplicon indicates that the sample was taken from a patient who is heterozygous for a deletion at the gene of interest. A ratio of about 1:1 indicates that the sample was taken from a patient who has a normal copy number of the gene of interest. A ratio of about 1.5:1 target amplicon to control amplicon indicates that the sample was taken from a patient who is heterozygous for a duplication at the gene of interest. A ratio of about 2:1 target amplicon to control amplicon indicates that the sample was taken from a patient who is homozygous for a duplication at the gene of interest. In another embodiment, the ratios can be correlated with the drug metabolizing phenotype of the patient.

In certain embodiments, the amplification and co-amplification steps can be any of the following types of reactions: the polymerase chain reaction (PCR), the ligase chain reaction (LCR), the rolling circle reaction, the strand displacement amplification (SDA) reaction, the nucleic acid sequence based amplification (NASBA) reaction, the transcription-based amplification system (TAS) reaction, the self-sustained sequence replication system (3SR) reaction, the Qβ replicase amplification system (Qβ) reaction, the real-time PCR reaction, and the Pyrosequencing™ reaction.

In another embodiment, the amplification and co-amplification steps use the polymerase chain reaction (“PCR”). The PCR reaction may include addition of 1M betaine. Additionally, the PCR is conducted with a single set of primers. In a particularly preferred embodiment, the forward primer of the pair has the sequence set forth in SEQ ID NO: 1, and the reverse primer of the pair has the sequence set forth in SEQ ID NO: 2. One primer of the pair may be labeled with an affinity moiety, preferably biotin. In yet another embodiment, the PCR reaction is conducted at an annealing temperature of 72° C. The PCR may include between about 25 and 45 cycles, but is preferably conducted for about 35 cycles.

In yet another embodiment, the gene of interest and the control gene are selected from the members of the cytochrome P450 gene family. More particularly, the control gene is a paralogous gene. More preferably, the gene of interest is the CYP2D6 gene and the control gene is the CYP2D8 gene.

In another preferred embodiment, the method is practiced in conjunction with an electronically addressable microchip. In this embodiment, the target and control amplicons may be electronically hybridized to probes, which may be bound to a support. In some embodiments, a permeation layer may be disposed above the electrodes in the microchip.

In still another embodiment, the detection step involves electronically or passively hybridizing a first reporter probe to the target amplicons and a second reporter probe to the control amplicons. The first and second reporters preferably have different labels to facilitate easy and simultaneous detection of the probes. In a preferred embodiment, the reporters are labeled with different fluorophores.

In a preferred embodiment, the detection step involves use of discriminators specific for the target amplicons and the control amplicons that have been bound to the probes on the microarray. In this instance, labeled universal reporters are subsequently hybridized to the discriminators such that the relative amounts of target and control amplicons can be ascertained. A plurality of universal reporters may be used in the practice of the invention, each bound to its own label to allow detection of several target and control sequences simultaneously.

### BRIEF DESCRIPTION OF THE DRAWINGS

FIG. 1 is a schematic example demonstrating that a single pair of primers can hybridize to common sequences in the CYP2D6 and CYP2D8 genes.

FIG. 2A is a schematic demonstrating that ratio information can be obtained by analyzing the relative intensities of two different fluorescent labels representing the presence or absence of large mutations or deletions in the CYP2D6 and CYP2D8 genes.

FIG. 2B shows the results of the method of the invention provides ratios that correlate with samples having known genetic duplications and deletions.

FIG. 3 shows the results of a PCR amplification reaction conducted on a sample containing a CYP2D6 gene duplication, and the results of a PCR amplification reaction conducted on a sample containing a CYP2D6 gene deletion, the amplification providing 3.5 kb and 3.2 kb products, respectively.

FIG. 4 shows the results of selective hybridization of the CYP2D6 gene product specifically to the CYP2D6 discriminator probe.

FIG. 5 shows the results of modifying the annealing temperature in the amplification step.

FIG. 6 shows the results of conducting the method with and without the addition of betaine to the amplification step. The upper panel shows the results conducted without the addition of betaine, while the lower panel shows the results with the addition of betaine.

FIG. 7 shows the results of a repeat of the experiment conducted in FIG. 6. The upper panel shows the results conducted without the addition of betaine, while the lower panel shows the results with the addition of betaine.

FIG. 8 shows the results of an experiment varying the concentration of betaine used in the amplification step with samples containing a duplicated CYP2D6 gene, a deleted CYP2D6 gene, and a wild-type CYP2D6 gene.

FIG. 9 shows the results of an experiment varying the number of PCR cycles within the amplification step.

FIG. 10 shows the results of an experiment varying the amount of genomic DNA within the biological sample.

FIG. 11 shows a comparison of the results of the method of the present invention with the results obtained by Long-Range PCR.

FIG. 12 shows the result of conducting the method using real-time PCR as the amplification method.

### DETAILED DESCRIPTION OF PREFERRED EMBODIMENTS

The present invention provides a method of detecting large mutations and deletions using a co-amplification control and an assay system in a high throughput format. In this method, specific regions of chromosomal DNA, which will also be referred to herein as genetic loci of interest or target amplicons, in biological samples are amplified simultaneously with other specific regions of chromosomal DNA, which will also be referred to herein as control genetic loci or control amplicons. The target and control amplicons may be analyzed in an assay device comprising a microchip that facilitates identification of specific markers. The target and control amplicons are analyzed in the assay to simultaneously determine the presence or absence of mutations, which can be insertions, deletions, duplications, repeats, transpositions, translocations, rearrangements and the like, to determine whether the mutation is present in one or both of a patient's chromosomes. The following definitions are used herein to describe the several embodiments of the invention.

An “amplicon” is an amplified polynucleotide sequence derived from a primer in an amplification reaction wherein a selected sequence is reproduced under reaction conditions that extend a primer sequence by sequential addition of nucleotides to encompass a target sequence.

“Amplification” refers to the process by which a region of a polynucleotide sequence is copied and expanded into a large number of amplicons. The polynucleotides contained in the patient samples may be amplified by in vitro methods, such as for example, polymerase chain reaction (PCR), the ligase chain reaction (LCR), rolling circle, strand displacement amplification (SDA), nucleic acid sequence based amplification (NASBA), the transcription-based amplification system (TAS), the self-sustained sequence replication system (3SR), the Qβ replicase amplification system (Qβ), real time PCR, and Pyrosequencing™.

Pyrosequencing, developed by Biotage AB (Uppsala, Sweden), is a non-electrophoretic, real-time DNA sequencing method in which a primer is hybridized to a single-stranded PCR template in the presence of DNA polymerase, ATP sulfurylase, luciferin and apyrase. Deoxynucleotide triphosphates (dNTPs) and adenosine 5′ phosphosulfate are then sequentially added to the reaction mixture. DNA polymerase catalyzes the incorporation of the dNTPs into the DNA strand, provided that there is complementarity to the template strand. Every time a dNTP is incorporated, pyrophosphate is emitted in a quantity equivalent to the incorporated nucleotide. ATP sulfurylase then quantitatively converts pyrophosphate to ATP, which drives the luciferase-mediated conversion of luciferin to oxyluciferin that subsequently generates visible light detectable by a camera visualized as a peak in Biotage's Pyrogram™ software. The peak heights in the resulting Pyrogram™ are directly proportional to the emitted light from the Pyrosequencing™ reaction, which allows quantitative analysis of, among other things, allele frequency in a mixed sample populations.

The Qβ replicase amplification system, which was developed by Epicentre Biotechnologies (Madison, Wis. USA) is an RNA-directed RNA polymerase responsible for replication of the Q-Beta RNA genome. The Qβ enzyme uses various RNA molecules as templates, including sub-genomic variant RNA molecules, such as midivarient RNAs. The replicase is a heterotetramer comprising a virally encoded subunit in addition to three host proteins: ribosomal protein SI, and elongation factors Tu and Ts. The enzyme also uses non-natural templates, such as poly(rC), RNA primed with an oligonucleotide or RNA molecules containing terminal C residues.

The term “biological sample,” as is well known to those of skill in the art, refers to any combination of bodily fluids, such as, for example, blood, urine, serum, lymph, saliva, and semen (all of which may be mammalian or non-mammalian); research samples; genomic DNA, RNA, or mRNA obtained from any source, such as, for example, bacteria, viruses, cells or tissues. The biological sample may comprise individual cells, primary cells or cell lines. Experimental manipulations may be conducted on the biological sample prior to its use in the present invention.

“Blockers” are polynucleotides that hybridize specifically to polynucleotide sequences, usually amplicons, and that are designed to prevent binding by wild-type and mutant discriminator probes.

“Complementary” refers to the topological compatibility or matching together of interacting surfaces of two oligonucleotides, i.e., a probe sequence and a target sequence. Thus, the two oligonucleotides can be described as complementary, and furthermore, the contact surface characteristics are complementary to each other. A first oligonucleotide, i.e., a probe, is complementary to a second oligonucleotide, i.e., a target, if the nucleotide sequence of the first oligonucleotide is identical to the nucleotide sequence of the oligonucleotide binding partner of the second. Thus, the oligonucleotide whose sequence 5′-TATAC-3′ is complementary to a oligonucleotide whose sequence is 5′-GTATA-3′. However, complementarity need not be perfect. There may be a number of single base-pair mismatches that may interfere with the hybridization between the first and second polynucleotides. If the number of mismatches is so large that no hybridization can occur between the polynucleotides, even under the least stringent conditions, then the two sequences are not complementary. Accordingly, the term “substantially complementary” means that a probe sequence is sufficiently complementary to the corresponding target sequence such that hybridization occurs under the selected reaction conditions.

“Detectable moiety” or “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, dioxigenin, haptens and proteins for which antisera or monoclonal antibodies are available, or nucleic acid molecules with a sequence complementary to a target. The label often generates a measurable signal, such as a radioactive, chromogenic, or fluorescent signal, that can be used to quantitate the amount of bound detectable moiety in a sample. The label can be incorporated in or attached to a primer or probe either covalently, or through ionic, van der Waals or hydrogen bonds, e.g., incorporation of radioactive nucleotides, or biotinylated nucleotides that are recognized by streptavidin. The label may be directly or indirectly detectable. Indirect detection can involve the binding of a second directly or indirectly detectable moiety to the detectable moiety. For example, the label can be the ligand of a binding partner, such as biotin, which is a binding partner for streptavidin, or a nucleotide sequence, which is the binding partner for a complementary sequence, to which it can specifically hybridize. The binding partner may itself be directly detectable, for example, an antibody may be itself labeled with a fluorescent molecule. The binding partner also may be indirectly detectable, for example, a nucleic acid having a complementary may be indirectly detectable, for example, a nucleic acid having a complementary nucleotide sequence can be a part of a branched DNA molecule that is in turn detectable through hybridization with other labeled nucleic acid molecules. (See, e.g., P. D. Fahrlander and A. Klausner, Bio/Technology (1988) 6:1165.) Quantitation of the signal generated by a label is achieved by known detection and measurement techniques, e.g., scintillation counting, densitometry, or flow cytometry.

A “discriminator” or “discriminator probe” is a polynucleotide that selectively binds to a polymorphic region of an amplicon, which region may or may not contain a mutation. Specific discriminator binding to a known, predetermined mutation is sometimes referred to herein as “querying.” Each different polymorphic region may be referred to as a “variant.” “Wild-type discriminators” bind to wild-type sequence, while “mutant” discriminators bind to variants including recognized mutations, or simple variants of the wild-type sequence that may be described as markers or polymorphisms. A “pair of discriminators” typically consists of the wild-type discriminator and the corresponding mutant discriminator for a specific polymorphism. Discriminators may consist of one that specifically binds to the sequence of a specific variant or of two polynucleotides that specifically bind, in direct apposition, to a contiguous sequence of a specific variant and are designed so that a first stabilizes the binding of a second by base stacking. The use of two polynucleotides and base stacking to obtain highly stable hybridization complexes capable of precise discrimination is described in Radtkey R. et al., Nucleic Acids Research, 28(7): i-vi (2000); and Yershov, G. et al., Proc. Nat'l Acad. Sci. USA, 93: 4913-18 (May 1996), which references are incorporated herein in their entirety.

“E-stripping” is electronic denaturation of double stranded polynucleotides or removal of hybridized polynucleotides.

“Heterozygous” means that one chromosome from a patient sample contains a mutant variant and the other chromosome contains the corresponding wild-type variant.

“Heterozygous ratio references” are polynucleotides that each bind specifically to one discriminator pair. Each heterozygous ratio reference contains one or more polynucleotides that bind to one pair of discriminators.

“Homozygous” means that both chromosomes from a patient sample contain a mutant variant or that both contain a wild-type variant. nucleotide sequence can be a part of a branched DNA molecule that is in turn detectable through hybridization with other labeled nucleic acid molecules. (See, e.g., P. D. Fahrlander and A. Klausner, Bio/Technology (1988) 6:1165.) Quantitation of the signal generated by a label is achieved by known detection and measurement techniques, e.g., scintillation counting, densitometry, or flow cytometry.

“Hybridizing” or “hybridizing specifically to” or “specific hybridization” or “hybridize to,” refers to the binding, duplexing, or hybridizing of a nucleic acid molecule preferentially to a particular nucleotide sequence under stringent conditions when that sequence is present in a complex mixture. As is known to those of skill in the art, hybridization generally depends on the ability of denatured oligonucleotides to anneal when complementary sequences are present in an environment having a temperature lower than their respective melting temperatures. The higher the desired degree of complementarity between a probe sequence and a target sequence, the higher the relative temperature that may be used. Accordingly, relatively higher temperatures tend to make the reaction conditions more stringent, whereas lower temperatures tend to make the reaction conditions less stringent. Stringency conditions are more fully explained in Current Protocols in Molecular Biology, Ausubel, et al. Several hybridization conditions may be used with the present invention, including high, moderate, and low stringency conditions. Detailed explanations of these conditions may be found, for example, in Sambrook, et al., Molecular Cloning: A Laboratory Manual, 2^{nd} ed, 1989. Stringent conditions are sequence-specific and will depend on the environment in which the hybridization is conducted. Longer sequences hybridize more specifically at higher temperatures. Stringent conditions are those in which the salt conditions are less than about 1.0M sodium ion, typically between about 0.01 to 1.0M at pH 7.0 to 8.3, and the temperature is at least about 30° C. for shorter probes (e.g., 10-50 nucleotides) and at least 60° C. for longer probes (e.g., more than 50 nucleotides) in an aqueous hybridization environment. Stringent conditions may also be obtained through the addition of helix destabilizing agents, such as formamide, to the hybridization reaction.

“Localized” means that a composition, e.g., an oligonucleotide, is concentrated at a test site to a level greater than in ordinary solution. In embodiments utilizing electronically addressable microchips, the term refers to a concentration of a composition, e.g., an oligonucleotide, which is greater than would occur through passive hybridization. In the context of an amplicon localized at a test site, the amplicon has a greater concentration achieved through chemical, electrical, or biochemical reaction, as opposed to mere selective placement at the test site.

The terms “mutation” or “polymorphism” describe nucleotide sequences that vary from a wild-type sequence by a known parameter such that the distinction can be interrogated with a discriminator probe. In typical usage, “mutation” usually refers to a variant of the wild-type sequence that is correlated to disease. A polymorphism may also be a mutation, but may also refer to a difference from the wild-type sequence that has no known correlation to disease. Both mutations and polymorphisms may broadly be described as “markers” for a disease or a marker may simply represent an identifiable sequence in comparison to wild-type. Markers, mutations, or polymorphisms may be deletions, substitutions, repeats, transpositions, translocations, insertions, rearrangements, etc. Generally, sequence variations present at frequencies less than 1% are considered mutations, while those present at higher frequencies are considered polymorphisms.

An “oligonucleotide” is a polymer of nucleotides. As is well known to those of skill in the art, several modifications to the sugar-phosphate backbone of the oligonucleotide to increase the stability of the oligonucleotides under the conditions used to practice the presently described invention. Oligonucleotides may be single-stranded, double-stranded or contain portions of both. The oligonucleotide may comprise DNA, RNA or a hybrid mixture of both. The term oligonucleotide also refers to non-naturally occurring structures analogous to nucleotides, such as, for example, peptide nucleic acids (PNA). The terms “oligonucleotide” and “polynucleotide” are used interchangeably herein.

A “primer” refers to a polynucleotide that is capable of specifically hybridizing to a designated polynucleotide template and providing a point of initiation for synthesis of a complementary polynucleotide under synthesis inducing conditions i.e., in the presence of nucleotides, a complementary polynucleotide template, and an agent for polymerization, such as DNA polymerase. A primer is typically a single-stranded deoxyribonucleic acid, but a wide variety of synthetic and naturally occurring primers are useful. A primer is complementary to the template to which it hybridizes to serve as a site for the initiation of synthesis.

A “reflex test” occurs when a positive genotyping result requires an additional test.

A “reporter” refers to a polynucleotide that is capable of specifically hybridizing to a designated sequence of another polynucleotide. A reporter contains a label. A probe specifically hybridizes to a target complementary polynucleotide, but need not reflect the exact complementary sequence of the template. In such a case, specific hybridization of the probe to the target depends on the stringency of the hybridization conditions. Probes can be labeled with, for example, chromogenic, radioactive, chemiluminescent, enzymatic, colorimetric, or fluorescent moieties (alone or in combination) and used as detectable moieties.

“Screening” is a step in the assay during which it is determined whether or not a sample contains any one of a group of polymorphisms (usually mutations) that are known to be related to disease. If a sample tests positive, the presence of a mutation within the subset is indicated and the sample can then be further analyzed or “genotyped.” “Genotyping” refers to determining whether a patient sample is homozygous for wild-type, homozygous for a particular mutant variant, or heterozygous for a particular variant.

The terms “selective for” or “selectively hybridize to” describe differential reactivity between wild type and a mutant variant of a probe in a hybridization reaction with a complementary sequence of an amplicon. A mutant discriminator probe is selective for a specific known polymorphism or mutation such that hybridization does not occur to a wild-type sequence. Similarly, a wild-type discriminator probe is selective for the wild-type sequence such that hybridization only occurs to the wild-type sequence and not to a polymorphism or mutation.

The term “stringent conditions” refers to conditions under which a probe will hybridize preferentially to its target subsequence, and to a lesser extent to, or not at all to, other sequences. “Stringent hybridization” and “stringent hybridization wash conditions” in the context of nucleic acid hybridization experiments, such as Southern and Northern hybridizations, are sequence dependent, and are different under different environmental parameters. An extensive guide to the hybridization of nucleic acids is found in Tijssen (1993) Laboratory Techniques in Biochemistry and Molecular Biology—Hybridization with Nucleic Acid Probes, part I chapter 2 “Overview of principles of hybridization and the strategy of nucleic acid probe assays,” Elsevier, N.Y. Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe.

A “universal reporter” refers to a polynucleotide that 1) possesses a moiety (e.g. a nucleotide sequence) that interacts with a series of nucleotide sequences, each in the series having the same moiety complementary to the universal reporter and a region specific for differing genetic loci, and 2) possesses a detectable moiety.

Device for Detecting the Presence of Large Insertions and Deletions

Several embodiments of the present invention utilize an electronically addressable microchip device. Many components of this device are described in the following patent applications and issued patents, which are specifically incorporated herein by reference:

Application Ser. No. 09/671,594, filed Sep. 27, 2000, entitled “ELECTRONIC SYSTEMS, COMPONENT DEVICES, MECHANISMS, METHODS, AND PROCEDURES FOR MACROSCOPIC AND MICROSCOPIC MOLECULAR BIOLOGICAL REACTIONS, ANALYSES AND DIAGNOSTICS”, which is a continuation-in-part of application Ser. No. 08/986,065, filed Dec. 5, 1997, entitled “METHODS AND PROCEDURES FOR MOLECULAR BIOLOGICAL ANALYSIS AND DIAGNOSTICS”, now issued as U.S. Pat. No. 6,051,380, which is a continuation-in-part of application Ser. No. 08/855,058, filed May 14, 1997, entitled “METHODS FOR ELECTRONIC FLUORESCENT PERTURBATION FOR ANALYSIS AND ELECTRONIC PERTURBATION CATALYSIS FOR SYNTHESIS”, now issued as U.S. Pat. No. 6,048,690, which is a continuation-in-part of application Ser. No. 08/534,454, filed Sep. 27, 1995, entitled “APPARATUS AND METHODS FOR ACTIVE PROGRAMMABLE MATRIX DEVICES”, now issued as U.S. Pat. No. 5,849,486, which is a continuation-in-part of application Ser. No. 08/304,657, filed Sep. 9, 1994, entitled “AUTOMATED MOLECULAR BIOLOGICAL DIAGNOSTIC SYSTEM,” now issued as U.S. Pat. No. 5,632,957, which is a continuation-in-part of application Ser. No. 08/271,882, filed Jul. 7, 1994, entitled “METHODS FOR ELECTRONIC STRINGENCY CONTROL FOR MOLECULAR BIOLOGICAL ANALYSIS AND DIAGNOSTICS,” now issued as U.S. Pat. No. 6,017,696, and which is a continuation-in-part of Ser. No. 08/146,504, filed Nov. 1, 1993, entitled “ACTIVE PROGRAMMABLE ELECTRONIC DEVICES FOR MOLECULAR BIOLOGICAL ANALYSIS AND DIAGNOSTICS”, now issued as U.S. Pat. No. 5,605,662.

The microchip device described in the applications and patents includes an array of microlocations or test sites each associated with an electrode. The electrode may be overlaid with a permeation layer that separates the polynucleotides from the surface of the electrode. The device also includes an attachment layer to which molecules such as nucleic acids are bound. Specific binding entities such as affinity binding pairs are immobilized on the attachment layer. For example, streptavidin can be incorporated into the permeation layer, providing an affinity binding site for nucleic acids that have been derivatized with biotin. The amplification primers may be biotinylated such that the amplicons are comprised of amplified loci of a patient sample and a first member of a binding pair wherein the second member of the binding pair is integral with the microchip. Charged molecules are electronically addressed to a specific test site by biasing the electrode underlying the test site with a charge opposite that of the target molecule. This process also results in localization of the molecule at the test site. In addition, the surrounding test sites may be biased with the same charge as the charged molecule such that the charged molecule is repelled.

In a preferred embodiment, the microchip device is coupled to a reader that detects signal generated by the labels attached to universal reporters that are hybridized to discriminator probes at the various test sites. In a particularly preferred embodiment, the reader detects at least two discrete wavelengths of fluorescent light generated by fluorescent labels incorporated into the universal reporters. The reader may be comprised of a discrete light source and a detector designed to detect a signal from the interaction between light from the source and a label used in the assay. Alternatively, the reader may obtain an image of the device during the assay, followed by image analysis to determine the results of the assay. In either embodiment, the reader detects the signal(s) generated by the reporter label(s) and determines intensity as well as a comparative value compared to like signals generated by the same label, or distinct signals generated by a different label or combination of labels. Where labeling by different reporters yields different signals, such as different fluorescent wavelengths, the detector measures the relative strengths of the signals at one or more locations, such as the test sites of the microchip, and may translate any of these detections into a signal for further processing by electronic means or through computer software that manipulates the signal to generate a data report of the results of the assay. In a particularly preferred embodiment, the microchip is also coupled to a loader capable of transferring sample from one container, such as a microtiter plate, to the microchip device and is capable of transferring members of the reagent set of the invention to the microchip.

### Method of Analysis

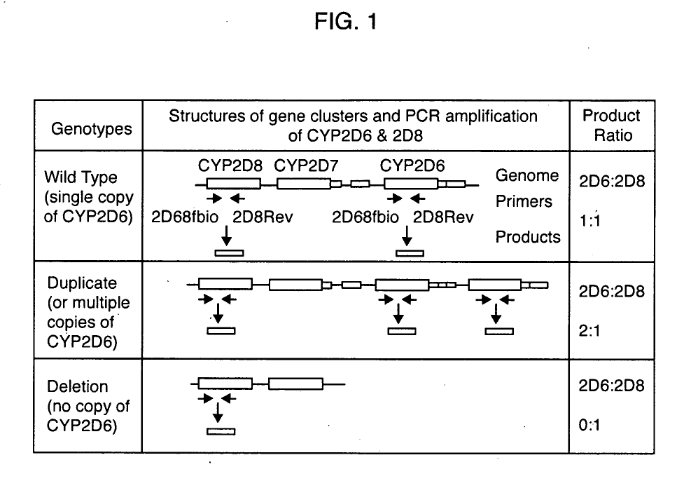

The method of the present invention analyzes the changes in copy number of a genetic loci of interest, for example, the CYP2D6 gene, to produce test amplicons that are normalized to a control genetic loci, for example, the CYP2D8 gene, to produce control amplicons. The control genetic loci has a copy number that remains stable in genome, as depicted in FIG. 1. In the preferred embodiment, the control genetic loci comprises a paralogous gene.

In a preferred embodiment, the method of the present invention utilizes a single pair of primers to accomplish a duplex amplification from both the CYP2D6 and CYP2D8 genes, where the paralogous CYP2D8 gene, whose copy number never changes, serves as an internal reference control. The use of such an endogenous gene, which is preferably a paralogous gene, as an internal control for normalization allows gene deletion and duplication analysis to be performed independent of the amount of gDNA template present in the biological sample from the patient.

For example, the target amplicon and control amplicon ratio for the two genes will always be about 1:1 in wild-type samples. Similarly, the target amplicon and control amplicon ratio will vary according to copy number of the genetic loci of interest in situations where mutant samples are analyzed. Importantly, the ratio will remain constant without regard to the amount of gDNA initially obtained form the patient biological sample. Theoretical calculations of the ratio of target and control amplicons are expressed herein as whole number, i.e., 1:1, 1.5:1, 2:1, etc. However, the experimental quantification of these ratios are often expressed in non-whole numbers. (See, e.g., Tables 1A and 1B.) Accordingly, the experimental values obtained as a result of practicing the invention are approximate and referred to herein as, for example, “a ratio of about 1:1” to reflect this difference between the theoretical and experimentally derived values.

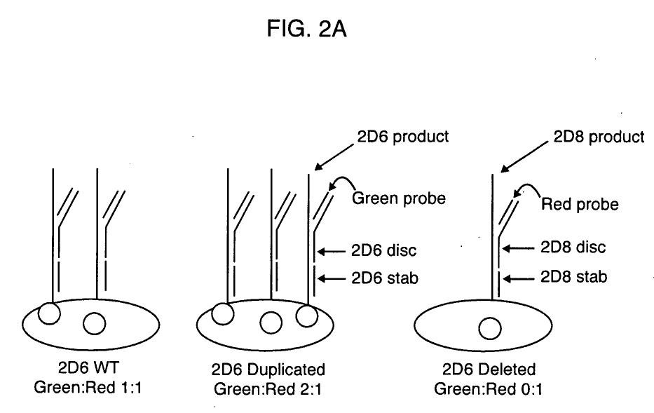

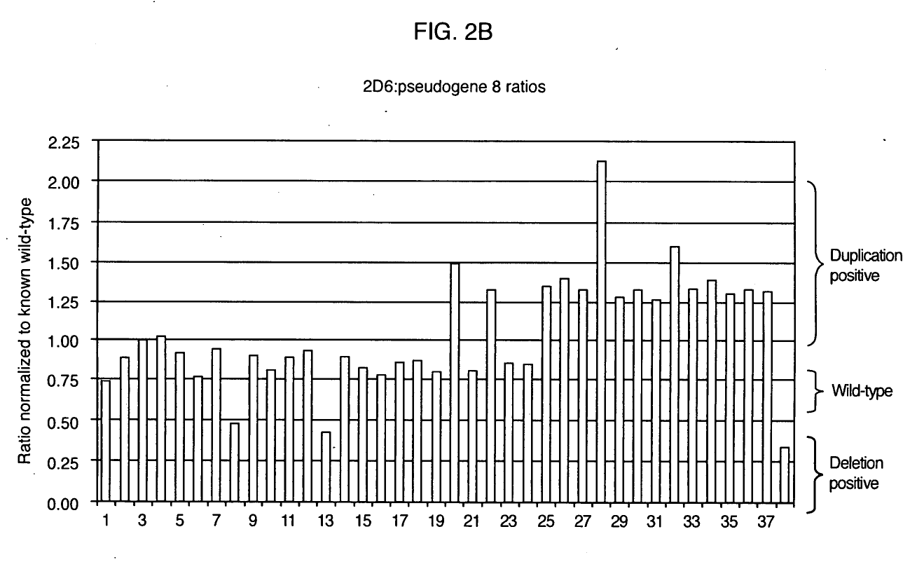

Using the same primers to amplify both the genetic loci of interest and the control loci has the benefit of maintaining similar amplification kinetics for the two genes increasing the likelihood that the quantity of target amplicons and control amplicons accurately represent the gene ratios present in the genome. The CYP2D6 and CYP2D8 genes share over 93% sequence homology, thereby increasing the opportunity to locate appropriate sequences that could be used for the primer pair. Indeed, in one embodiment, the primer pair has produced PCR products of equal length (300 bps) from the two genes, thereby further minimizing the kinetic differences between amplification of the genetic loci of interest and the control loci. Despite the similarity in the sequence of the target amplicon and the control amplicon, the differences are still distinguishable by the discrimination probes used in the NanoChip® Electronic Microarray, as shown in FIG. 2.

An underlying principle of the method of the present invention is that product yields of all target genes are linearly correlative to the copy numbers of target genes in the biological sample from the patient. That is, the molecular ratio of target and control amplicons achieved as a result of the amplification steps accurately represents the molecular ratio of target and control genes in genome. Although a linear relationship between gene copy and product yield in PCR has been repeatedly demonstrated and is the foundation for quantitative competitive PCR, achieving such linearity simultaneously for multiple genes, i.e., the test and control genes, can be challenging due to the exponential nature of PCR, wherein small variations in amplification efficiency may lead to dramatic changes in product yields. In addition, the amount of amplified product produced, whether target or control amplicons, reaches a plateau during later stages of PCR. These characteristics can obscure difference in the levels of target and control amplification, thereby introducing bias into the product ratio.

As a result, studies have shown that even using the same primers to co-amplify two genes having equal copy number in genome, e.g., the wild type samples, amplification efficiency for the two genes was seldom equal, potentially leading to a biased and inconsistent product ratio from sample to sample or even among repeats of the same sample. Under those conditions, it is very difficult to conduct an accurate gene copy analysis. However, equivalent co-amplification for two genes resulting in a consistent product ratio of about 1:1 in wild-type samples can be achieved through a combination of increased primer annealing temperature (to 72° C.) and addition of betaine (at 1M) to the PCR amplification.

The number of amplification cycles also affects the analysis. The number of cycles for optimal amplification is 35 cycles. Alternatively, amplification reactions reaching the second half of exponential phase, as verified by real time PCR, have provided suitable amplification conditions. Although reducing the cycling number to 25 cycles (corresponding to an early exponential phase) did not dramatically alter the linearity of the amplification and still allowed satisfactory performance of the analysis, the yields of test amplicon and control amplicon were remarkably low (about 10 fold lower than that from the 35 cycle reactions). Increasing the cycling number to 45 cycles, thereby allowing the amplification to extend into the plateau phase, obscured the difference in product yield for mutant samples in a manner sufficient to make the analysis less robust. With a 35-cycle reaction, a template level ranging from between about 12.5 ng to 50 ng gDNA (or about 0.5 ng to 2 ng/μl in a 25 μl reaction) allows all amplifications to fall within the right stage (the exponential phase) for genotype analysis.

The method of the present invention was tested against ‘known’ samples whose genotypes were determined according to the Long-Range PCR method. The results from each method matched completely (100%). The test amplicon and control amplicon ratios (as demonstrated by R:G ratio, obtained by using differently labeled reporters for test and control amplicons, respectively) between the CYP2D6 and CYP2D8 genes from the experimental analysis were different from the predicted values shown in FIG. 1. This discrepancy is likely due to the fact that the test and control amplicon ratios in FIG. 1 were calculated from a single haplotype, while the actual test and control amplicon ratios were analyzed from a fill genome containing two haplotypes. Tables 1A and 1B show the test and control amplicon (and gene) ratios calculated from full genomes demonstrating the well-matched coincidence between calculated and experimental ratios.

TABLE 1A
Calculated CYP2D6:CYP2D8 product ratio from full genotypes

| | Gene copy | Calculated | Actual assay * |
|---|---|---|---|
| | ratio | ratio | ratio |
| Genotype | 2D6:2D8 | Green:Red | Green:Red |
| Wt/wt | 2:2 | 1:1 | 1:1 ^{a} |
| Wt/del | 1:2 | 1:2 | 1:2.2 ± 0.3 ^{b} |
| Wt/dup | 3:2 | 1.5:1 | 1.53 ± 0.04:1^{c} |
| Del/del | 0:2 | Red only | n/a |
| Del/dup | 2:2 | 1:1 | n/a |
| Dup/dup | 4:2 | 2:1 | n/a |

* Mean and std values calculated from N = 4 ^{a}, 6 ^{b} and 3^{c} samples

TABLE 1B
Experimental Results for CYP2D68 haplotype analysis
on a NanoChip ® microarray

| | | | Ratio | Determined | Known* |
|---|---|---|---|---|---|
| Sample | Red | Green | (R:G) | Haplotype | Haplotype |
| #55 | 188 | 317 | 1:1.69 | Dup | Dup |
| #68 | 48 | 100 | 1:2.08 | Dup | Dup |
| #786 | 98 | 148 | 1:1.51 | Dup | Dup |
| #841 | 70 | 105 | 1:1.49 | Dup | Dup |
| #27 | 459 | 241 | 1.91:1 | Del | Del |
| #40 | 440 | 219 | 2.01:1 | Del | Del |
| #45 | 121 | 70 | 1.74:1 | Del | Del |
| #825 | 412 | 164 | 2.51:1 | Del | Del |
| #826 | 562 | 300 | 1.87:1 | Del | Del |
| #828 | 182 | 99 | 1.84:1 | Del | Del |
| #842 | 202 | 205 | 1:1.01 | Wt | Wt |
| #840 | 103 | 109 | 1:1.06 | Wt | Wt |
| #201 | 125 | 112 | 1.11:1 | Wt | Wt |
| #202 | 147 | 147 | 1:1 | Wt | Wt |

*According to the Long-Range PCR method (Johansson et al., 1996; Lovie et al., 1996).

An enlarged product ratio from sample No. 68 suggests the subject has multi-duplicated CYP2D6 genes in the genome. This consistency between theoretical and actual analysis supports the logistics of our hypothesis and analysis for CYP2D6 gene deletion and duplication.

The method of the present invention was also tested against samples whose duplication and deletion status known. Specifically, the method of the present invention was compared to results obtained by using the Roche AmpliChip CYP450 Test (Roche Diagnostics, Basel, Switzerland). The AmpliChip CYP450 Test uses microarray technology to detect genetic variations in CYP2D6 and CYP2C19 genes and is capable of providing associated predictive phenotype information. FIG. 2B shows that the method of the present invention yielded ratios that were corroborated by samples containing known duplications and deletions.

### Base Stacking Embodiment

In another preferred embodiment, the method of the present invention utilizes the base stacking techniques described in U.S. Pat. No. 6,753,148, which is hereby fully incorporated by reference. In the base stacking technique, the following sequence can be used as the CYP2D6 stabilizer: 5′CCATCCACCACCCACTCCAACCCTATGCTCC3′ (SEQ ID NO: 7). Similarly, the following sequence can be used as the CYP2D8 paralogous gene stabilizer: 5′CCCTCCGCCGCCCACTCCAACCCTGTGCTTT3′ (SEQ ID NO: 8). In embodiments employing the base stacking system, a variety of probes can be designed in and around the set of polymorphisms following procedures that are known to those of skill in the art. For example, one suitable probe for the CYP2D6 gene has the sequence 5′GCATTAGCCCCA3′ (SEQ ID NO: 11); a suitable probe for the CYP2D8 paralogous gene has the sequence 5′TTGGCCCCG3′ (SEQ ID NO: 12). The present invention is not limited to the specific sequences disclosed herein, but rather may be selected using techniques well known to those of skill in the art. The probes used in the base stacking system may be labeled directly, for example, with a fluorophore. Alternatively, the base stacking system may be used in conjunction with a series of discriminator probes and universal reporters, as has been previously described in, for example, U.S. Pat. No. 6,753,148 and U.S. Patent Publication No. 2004/0146880, which is also hereby fully incorporated by reference.

Additional embodiments of the invention are described in the Examples below. Although the examples below are primarily directed to co-amplification of the CYP2D6 gene and its related pseudogenes, the invention is not intended to be so limited. For example, the invention may be successfully utilized with the CYP21B gene and its psuedogene CYP21A. Patients who lack the functional protein, 21-hydroxylase, encoded by the CYP21B gene may develop congenital adrenal hyperplasia. Accordingly, use of the present invention may lead to beneficial, early detection of this cancer.

### EXAMPLE 1

### Sample Preparation

Genomic DNA (gDNA) was prepared from human whole blood samples obtained from San Diego Blood Bank (San Diego, Calif., USA) using Qiagen Midi DNA Kit (Qiagen, Valencia, Calif., USA). The purified DNA was re-suspended in deionized dH₂O and stored at −20° C. until use. The AmpliTaq PCR kit was purchased from PE/Applied Biosystems (Foster City, Calif., USA), and the Expand Long Template PCR System was purchased from Roche Applied Science. All oligonucleotides were synthesized in the Integrated DNA Technologies (IDT, Coralville, Iowa, USA).

Sample Amplification

To analyze CYP2D6 gene duplication and deletion, a duplex PCR strategy was developed that amplifies 2 heterozygous products of equal product length (300 bps) from the CYP2D6 and CYP2D8 genes, respectively, with a single pair of primers that hybridize to the common binding sequences in the CYP2D6 and CYP2D8 genes, as shown in FIG. 1. The forward, but not the reverse, primers were biotinylated. The amplification was accomplished in 25 ul volume containing 1M betaine in addition to 1× buffer II (AmpliTaq kit, PE/Applied Biosystems, Foster City, USA), 3.5 mM MgCl₂, 200 μM of each dNTP, 0.2 μM of each primer, 50 ng gDNA and 0.511 of AmpliTaq Gold polymerase (5 U/μl). The amplification reaction included an initial heat denaturing step at 96° C. for 10 min, followed by 35 cycles of 95° C. for 20 sec and 72° C. for 30 sec. There was no final elongation step prior to completion of the reaction.

Genotype analysis on NanoChip® Electronic Microarray

Detection of CYP2D6 gene duplication and deletion from the above amplifications were performed on a NanoChip® Electronic Microarray following the standard SNP analysis procedures that have been described previously. In brief, the PCR reactions were de-salted on a Millipore MultiScreen PCR desalting plate (Millipore, Bedford, Mass., USA) and re-suspended in 10-fold volume of 50 mM histidine. The solutions were transferred to a 96-well microtiter plate and electronically loaded on the Nanogen Molecular Biology Workstation (MBW) Loader to a NanoChip® Electronic Microarray where the biotinylated strands of the amplicons (from both genes) attached to streptavidin in the permeation layer on the Electronic Microarray. The complementary non-biotinylated strands were washed off the Electronic Microarray. The attached amplicons (containing a mixture of the CYP2D6 and CYP2D8 gene products) on the Electronic Microarray were detected and discriminated by two fluorescently labeled probes through two discriminator oligonucleotides that selectively hybridize to the CYP2D6 and CYP2D8 gene products, respectively (green for CYP2D6 and red for CYP2D8, sequences of the discriminator and probe oligos, see Table 2; the corresponding SEQ ID NO is indicated below each corresponding sequence).

TABLE 2

| | | |
|---|---|---|
| Names | sequences | target to hybridize |

PCR primers:

| | | |
|---|---|---|
| 2D68fbio | 5′bio-cagggactgcgggagaccag-3′ | (CYP2D6 & CYP2D8 genes) |
| | (SEQ ID NO: 1) | |
| 2D68rev | 5′-CCTTCCCAGTTCCCGCTTTGTGC-3′ | (CYP2D6 & CYP2D8 genes) |
| | (SEQ ID NO: 2) | |
| 2D6rev | 5′-CCATAGCGCGCCAGGAACAC-3′ | (CYP2D6 gene) |
| | (SEQ ID NO: 3) | |
| 2D8rev | 5′-GTCCGTAGTGTGCCAGAAACAC-3′ | (CYP2D8 gene) |
| | (SEQ ID NO: 4) | |

Reporting oligos:

| | | |
|---|---|---|
| 2D6.disc | 5′-ctgagtccgaacattgagtttGCATTAGCCCcA-3′ | (CYP2D6 amplicon) |
| | (SEQ ID NO: 5) | |
| 2D8.disc | 5′-gcagtatatcgcttgacaCTTGGCCCCG-3′ | (CYP2D8 amplicon) |
| | (SEQ ID NO: 6) | |
| 2D6.stab | 5′ CCATCCACCACCCACTCCAACCCTATGCTCC3′ | (CYP2D6 amplicon) |
| | (SEQ ID NO: 7) | |
| 2D8.stab | 5′ CCCTCCGCCGCCCACTCCAACCCTGTGCTTT3′ | (CYP2D8 amplicon) |
| | (SEQ ID NO: 8) | |
| Green probe | 5′-CTCAATGTTCGGACTCAG-Alexas532 | (2D6.disc) |
| | (SEQ ID NO: 9) | |
| Red probe | 5′-TGTCAAGCGATATACTGC-Alexas647 | (2D8.disc) |
| | (SEQ ID NO: 10) | |

The fluorescent signals detected from the Electronic Microarray (on the MBW Electronic Microarray Reader) represent the yields of gene product (CYP2D6 or CYP2D8), while the ratio between the two fluorescent signals provides the information relating to CYP2D6 genotypes, either gene deletion or duplication, as depicted in FIG. 2.

Long-Range PCR on the GeneAmp® PCR System

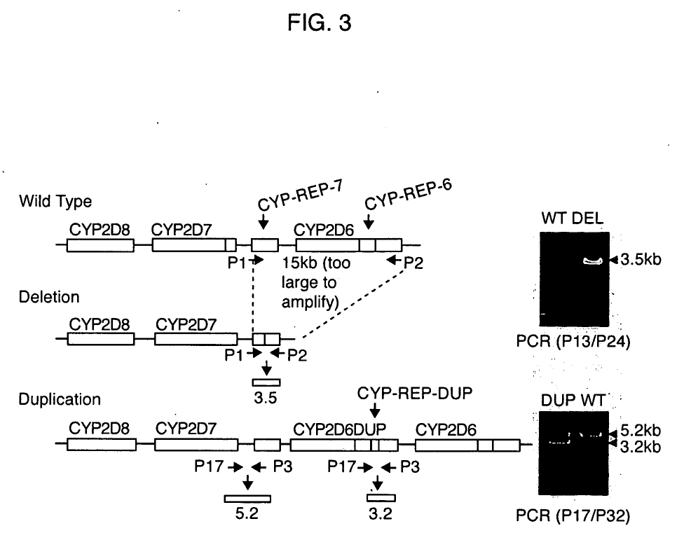

Two PCR amplifications were performed on each genomic DNA samples with the Long-Range PCR method (Roche Applied Science) on GeneAmp® PCR systems (9700 and 2700 models) to identify samples with CYP2D6 gene deletions or duplications according to the previous reports by Johansson et al. (1996) and Lovie et al. (1996). These references report that PCR on a sample that contain at least one additional copy of CYP2D6 gene (‘duplication’) produces a 3.5-kb product, while PCR on a sample that has fewer copies of the CYP2D6 gene (‘deletion’) produces a 3.2-kb product, as shown in FIG. 3. With the Long-Range PCR method using the primers reported previously by Johansson et al. (1996) and Lovie et al. (1996), four samples were identified that contain additional CYP2D6 genes (CYP2D6*×N, *2×N or *4×N alleles). In addition, five samples were identified that were lacking the full complement of genomic CYP2D6 genes (CYP2D6*5 allele). These samples of ‘known’ genotypes were used to verify the genotyping results from analysis on NanoChip® Electronic Microarray developed in this study.

### Example 2

### Specificity of Product Amplification and Discrimination

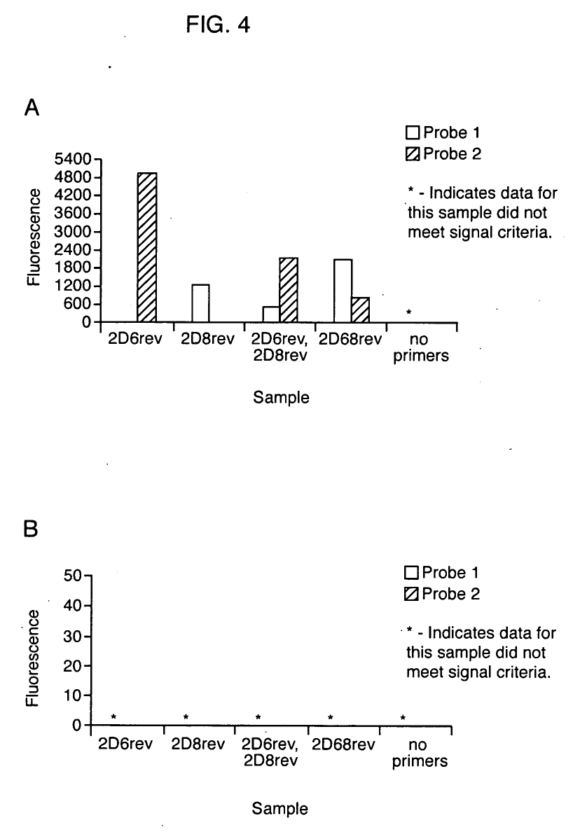

High specificity of genetic loci amplification and discrimination is important to the present invention. In order to verify sufficient specificity and discrimination, PCR was conducted to amplify the CYP2D6 and CYP2D8 genes separately, or simultaneously, using a common forward primer (2D68fbio) but different reverse primers, as shown in FIG. 3 and Table 1. All reactions proceeded as described previously. After loading onto a NanoChip® Electronic Microarray, the products were incubated under hybridization conditions with a reporter mix containing discriminator probes for both CYP2D6 and CYP2D8 gene products. As shown in FIG. 4, the genes were selectively amplified with designated primer sets. The CYP2D6 gene product hybridized only to the CYP2D6 discriminator probe and not to CYP2D8 discriminator probe, thereby producing a clean green signal for the CYP2D6 gene product on Electronic Microarray (the ‘2D6rev’ pad).

The converse is also shown in FIG. 4. The CYP2D8 gene product hybridized only to the CYP2D8 discriminator probe to produce a clean red signal for the CYP2D8 gene product on Electronic Microarray (the ‘2D8rev’ pad). When both genes were amplified in a reaction (with either a common reverse primer 2D68rev or a mixture of both reverse primers 2D6rev and 2D8rev to the two genes), their products hybridized to both discriminators (2D6.disc and 2D8.disc), respectively, to produce both green and red signals. Co-amplification of a genetic locus of interest and a paralogous gene control allows comparison of relative yields and facilitates determination of a ratio that can be used to classify an individual according to his ability to metabolize certain prescription drugs. The results demonstrates that discriminator probes for the CYP2D6 and CYP2D8 genes (2D6.disc and 2D8.disc) are highly specific to their respective targets and can be used for this analysis.

When the same amplification products were reported with discriminator probes that were specific for CYP2D7 gene product, which shares approximately 95% sequence homology with the CYP2D6 and CYP2D8 gene products, no CYP2D7 products were detected. This result indicates a highly specific amplification of CYP2D6 and CYP2D8 genes in the duplex reactions with the primer pair 2D68fbio and 2D68rev. Therefore, this pair of primers (2D68fbio/2D68rev), is capable of specifically and simultaneously amplifying both CYP2D6 and CYP2D8 genes.

Effect of PCR Annealing Temperature

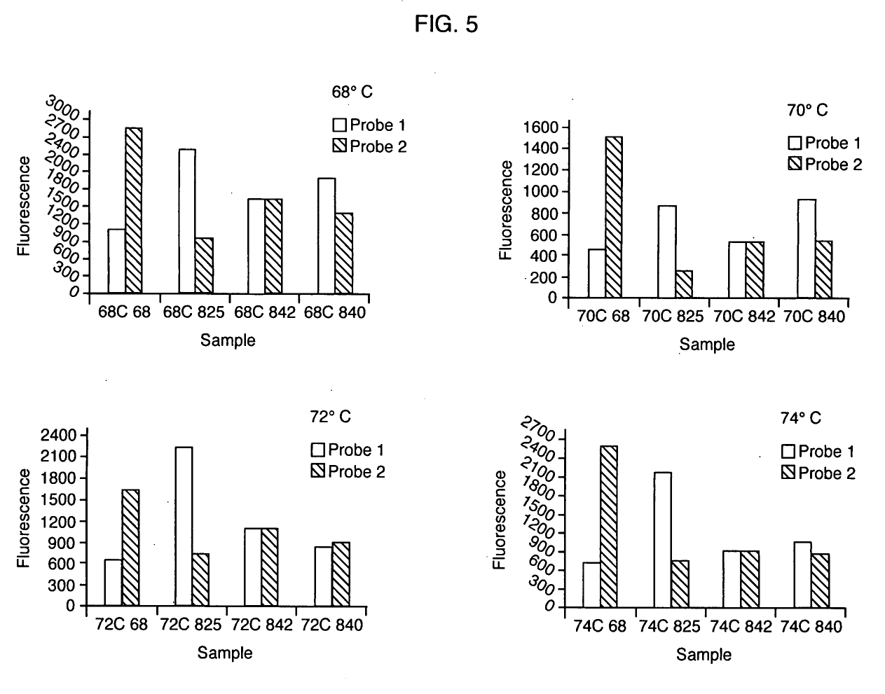

Tests with elevated annealing temperatures were performed to determine the optimal temperature to yield consistent amounts of target amplicons and control amplicons in order to facilitate quantifiable comparisons. Four sets of 16 total reactions for four genomic samples (2 mutant and 2 wild type samples) were prepared as described above (using the primer set 2D68fbio/2D68rev) and subjected to 4 amplification reactions, with annealing temperatures set to 68° C., 70° C., 72° C. and 74° C., respectively. Each amplification reaction was cycled for 35 times. FIG. 5 shows the amplification yields and green:red ratios for each of the four reactions. Although all four temperature conditions resulted in sufficiently quantifiable yields for mutant samples (No. 68 with duplicated CYP2D6 and No. 825 with deleted CYP2D6), i.e., producing largely different green:red ratios suitable for haplotyping, when the ratios were normalized with the wild type sample No. 842, only the PCR condition with annealing temperature at 72° C. produced consistent ratios for all wild type samples (Nos. 842 and 840). Amplification using an annealing temperature of 72° C. allowed random use of the wild type samples for appropriate normalization of mutant samples without significantly affecting the ratios in mutant samples. Increasing or decreasing the annealing temperature appeared to reduce the uniformity of wild type green:red ratios and affect the ability to make quantifiable comparisons between the hybridized target amplicons and hybridized control amplicons. Accordingly, a PCR protocol with annealing temperature of 72° C. and 35 cycles (between 95° C. for 20 sec and 72° C. for 30 sec) provided the optimal results.

Effect of Adding Betaine to the PCR Reaction

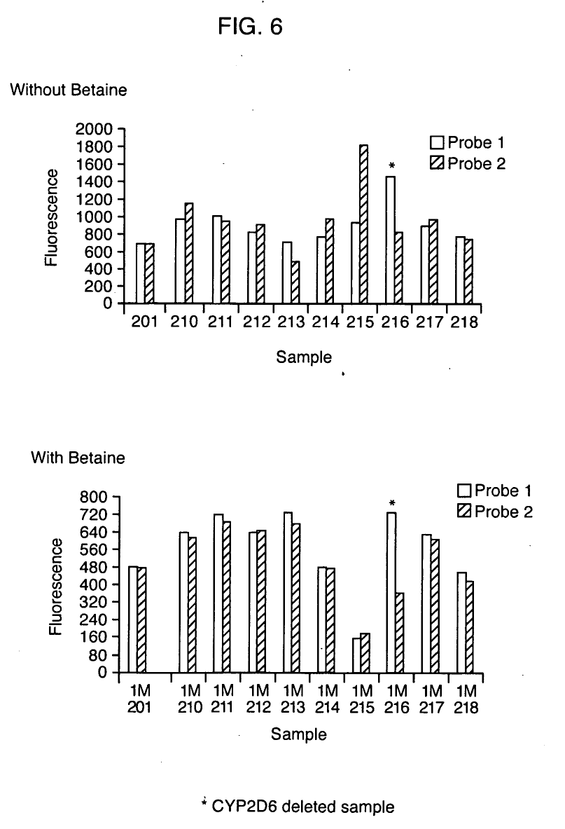

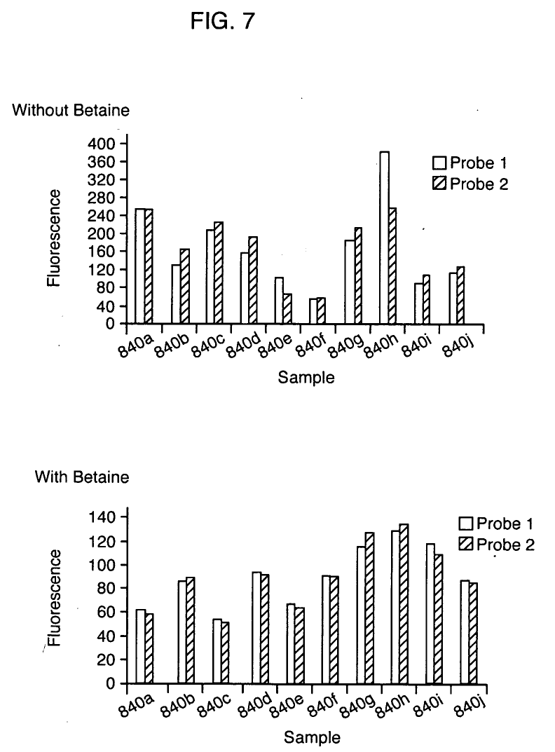

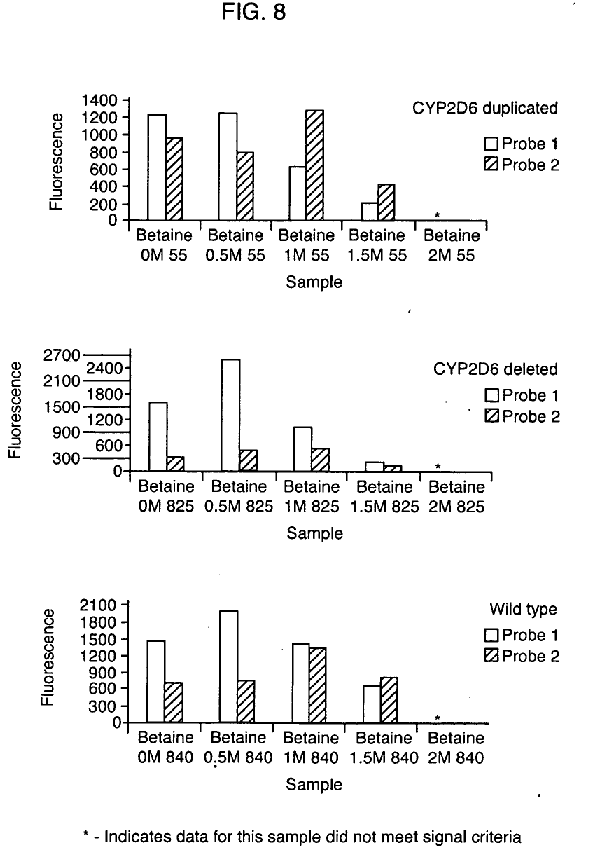

Although conducting PCR reactions using an annealing temperature of 72° C. helped to improve the uniformity of green:red ratios in wild type samples, the results were inconsistent. Specifically, uniformity was less than perfectly reproducible, either among wild type samples (FIG. 6) or among repeats from the same sample (FIG. 7). The addition of 1M betaine to amplification reaction under the conditions described previously greatly improved the uniformity of the green:red ratios as shown in FIGS. 6 and 7. Titrating the concentrations of betaine in the amplification reaction (between about 0 to 2.0M) yielded an optimal concentration of 1M betaine for all genotypes, i.e., capable of minimizing the product ratios in wild type sample to close to 1:1 while maximizing the ratios in mutant samples as shown in FIG. 8.

Effect of the Number of PCR Cycles

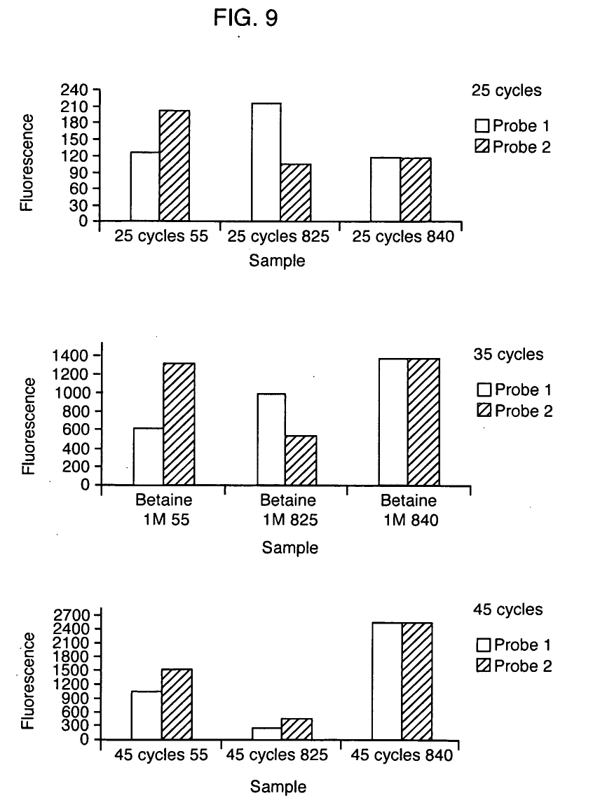

Studies were conducted to test the effect of cycling number in the PCR reaction on product ratio. A master reaction mix containing 1M betaine was prepared for nine amplification reactions to amplify a sample having a CYP2D6 duplication (No. 55), a CYP2D6 deletion (No. 825), and a CYP2D6 wild type (No. 840). The reaction conditions were held constant [96° C. 10 min, followed by (95° C. for 20 sec and 72° C. for 30 sec)] but cycled 25, 35, and 45 times, respectively. Although fewer cycles (25 cycles) tended to produce good discrimination ratios for genotype analysis, the lower cycle number produced lower amplification yield of product (about 10 fold less in a 35-cycle reaction). In contrast, more cycles (45 cycles) increased product yields but reduced the resulting ratios. As shown in FIG. 9, 35 cycles produced both a high product yield and a good discrimination ratio.

Effect of Genomic Template Level

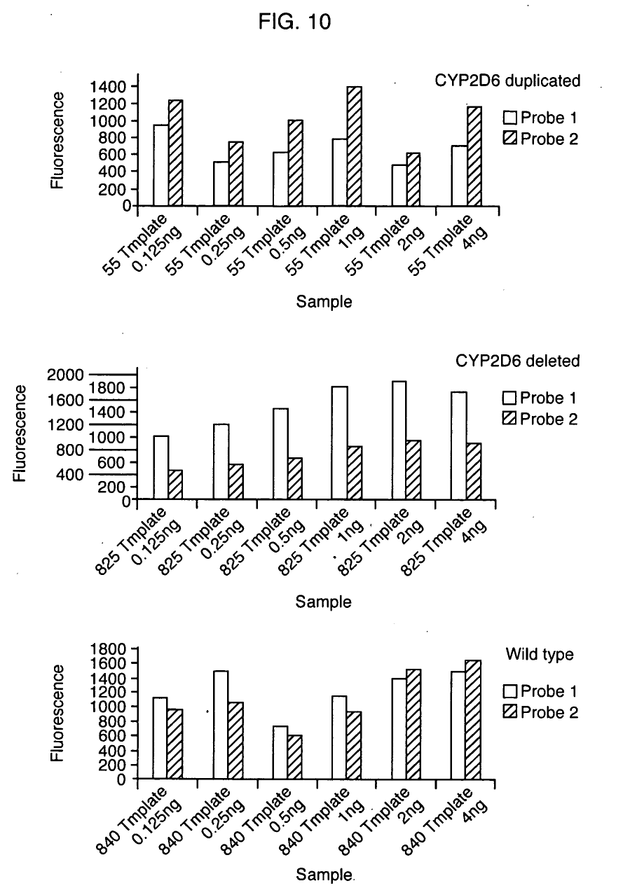

Using the conditions described previously, studies were conducted to examine the effect of the starting amounts of template on the analysis. A master reaction mix for 18 reactions was prepared and divided into 3 groups having 6 reactions each. Each reaction contained between about 0.125 ng and 4 ng/μl of gDNA (equivalent to between about 3.125 ng and 100 ng gDNA per 25 μl reaction) for the 3 samples No. 55, No. 825 and No. 840, respectively. FIG. 10 shows all reactions with the templates produced good amplifications and relatively consistent ratios closely matching the actual genotypes of the template samples. Nevertheless, reactions with template levels ranging from 0.5 ng/μl (12.5 ng per reaction) to 2 ng/μl (50 ng per reaction) produced homozygous discrimination ratios for all 3 genotype samples.

Comparison with Long-Range PCR

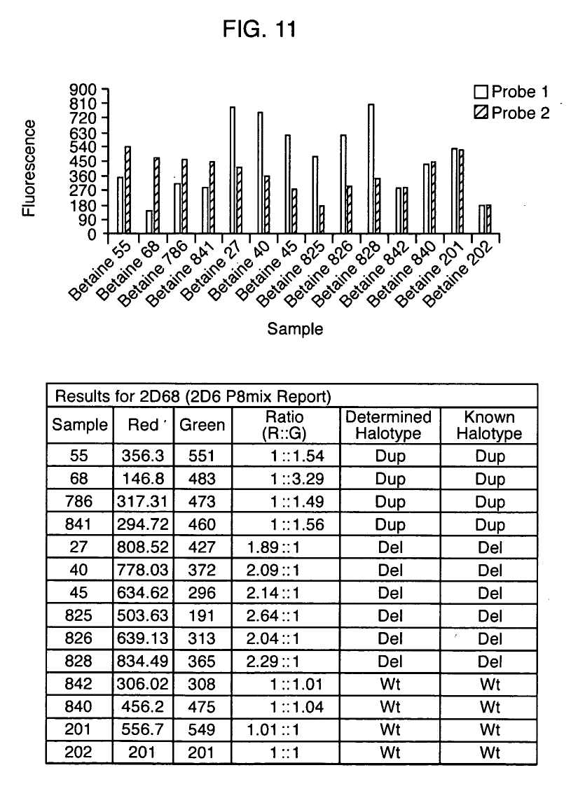

Using the optimized conditions previously described, an amplification analysis was conducted and compared against ‘known’ samples as determined by the Long-Range PCR method. FIG. 11 shows that the method described herein accurately matched results of genotypes determined by the Long-Range PCR method.

Use of Real-Time PCR as the Amplification Protocol

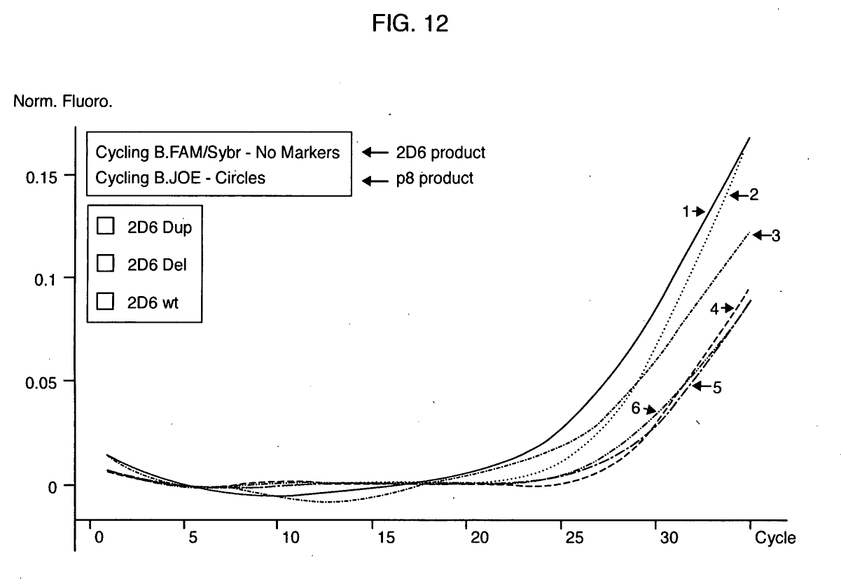

FIG. 12 shows the results of the method using real-time PCR as the amplification process, using the CYP2D6 and CYP2D6 pseudogene 8 genes as templates and probes incorporating molecular beacons. The 2D6 molecular beacon probe was 5′6-FAM-tgggacggTTCATGGCCACGCGCACGTGCCCGTCCCA-3′BHQ-1 (SEQ ID NO: 13) and the 2D6p8 molecular beacon probe was 5′6-TET-cgggacggTCCATGACCACGTCCATGTGTCCGTCCCG-3′BHQ-1 (SEQ ID NO: 14). Selection and design of the particular probe used is within the knowledge of one of ordinary skill in the art, and the present invention is not limited to the particular probes disclosed herein. Each 10 μl reaction mixture contained the following components:

| | |
|---|---|
| dH2O | 0.8 μl |
| 10x buffer (PE/ABI) | 1.0 μl |
| MgCl2 (25 mM) | 1.4 μl |
| dNTPs (10 nM each) | 0.2 μl |
| 2D68 fbio (10 μM) | 0.2 μl |
| 2D68Rev (10 μM) | 0.2 μl |
| 2D 6 probe (FAM, 1 μM) | 1.0 μl |
| 2D6p8 probe (TET, 2 μM) | 1.0 μl |
| Taq Gold (5 U/μl) | 0.2 μl |
| Genetic DNA (5 ng/μl) | 4.0 μl |

The real-time PCR cycling conditions were as follows:
hold at 96° C. for 10 min;
cycling (with 35 repetitions):
   step 1 at 95° C., hold for 20 sec
   step 2 at 68° C., hold for 30 sec
   step 3 at 72° C., hold for 20 sec; and
hold at 72° C. for 1 min.

The reaction was carried out in 0.1 ml tubes (Corbett Research, cat 3001-002) on Rotor-gene 3000 Four-Channel Multiplexing System (RG-3000™, Corbett Research). The real time signals were acquired in cycling step 2 (68° C.) with excitation source 470 nm and detection filter 510 nm for FAM dye (2D6 probe) and excitation source 530 nm and detection filter 555 nm for TET dye (2D6p8 probe), and analyzed by the RG-3000™ software. On the graph in FIG. 12, lines 2, 4 and 6 refer to CYP2D6 sequences from the duplicate, wild-type, and deletion samples, respectively. Similarly, lines 1, 3 and 5 refer to CYP2D6 pseudogene 8 sequences from the deletion, duplicate, and wild-type samples, respectively. As the graph in FIG. 12 shows, the wild-type melt curves (lines 4,5) tracked together, the duplication-positive samples (lines 1,6) tracked higher than the psuedogene 8 reference, and the deletion-positive sample (line 2) tracked lower than the reference.

Although the foregoing invention has been described in some detail by way of illustration and example for purposes of clarity and understanding, it may be readily apparent to those of ordinary skill in the art in light of the teachings of this invention that certain changes and modifications may be made thereto without departing from the spirit or scope of the appended claims.

What is claimed is:

1. A method for detecting a mutation in a genetic locus of interest in a biological sample, the method comprising:
(a) isolating genomic DNA from the biological sample;
(b) amplifying a portion of the genetic locus of interest from the biological sample to produce target amplicons;
(c) co-amplifying a portion of a control genetic locus from the biological sample to produce control amplicons, wherein the control genetic locus and the genetic locus are paralogous genes;
(d) detecting the amount of target amplicons relative to the amount of control amplicons; and
(e) determining a ratio of the relative amounts of target amplicons to control amplicons, wherein the ratio is indicative of presence or absence of the mutation in the genetic locus of interest.

2. The method of claim 1, wherein a ratio of about 0:1 target amplicons to control amplicons indicates that the biological sample is homozygous for a deletion of the genetic locus of interest.

3. The method of claim 2, wherein the ratio indicates that the biological sample was taken from a patient who is a poor metabolizer.

4. The method of claim 1, wherein a ratio of about 0.5:1 target amplicons to control amplicons indicates that the biological sample is heterozygous for a deletion of the genetic locus of interest.

5. The method of claim 4, wherein the ratio indicates that the biological sample was taken from a patient who is a poor, intermediate or extensive metabolizer.

6. The method of claim 1, wherein a ratio of about 1:1 target amplicons to control amplicons indicates that the biological sample has a normal gene copy number at the genetic locus of interest.

7. The method of claim 6, wherein the ratio indicates that the biological sample was taken from a patient who is a poor, intermediate, extensive or ultrarapid metabolizer.

8. The method of claim 1, wherein a ratio of about 1.5:1 target amplicons to hybridized control amplicons indicates that the biological sample is heterozygous for a duplication of the genetic locus of interest.

9. The method of claim 8, wherein the ratio indicates that the biological sample was taken from a patient who is a poor, intermediate, extensive or ultrarapid metabolizer.

10. The method of claim 1, wherein a ratio of about 2:1 target amplicons to hybridized control amplicons indicates that the biological sample is homozygous for a duplication of the genetic locus of interest.

11. The method of claim 10, wherein the ratio indicates that the biological sample was taken from a patient who is a poor, intermediate, extensive or ultrarapid metabolizer.

12. The method of claim 1, wherein the amplification step (b) and the co-amplification step (c) comprise a method selected from the group consisting of a polymerase chain reaction (PCR), a ligase chain reaction (LCR), a rolling circle reaction, a strand displacement amplification (SDA) reaction, a nucleic acid sequence based amplification (NASBA) reaction, a transcription-based amplification system (TAS) reaction, a self-sustained sequence replication system (3SR) reaction, a Qβ replicase amplification system (Qβ) reaction, a real-time PCR reaction, and a Pyrosequencing™ reaction.

13. The method of claim 1, wherein the amplification step (b) and the co-amplification step (c) include PCR.

14. The method of claim 13, wherein the PCR is performed in the presence of 1 M betaine.

15. The method of claim 13, wherein the amplification step (b) and the co-amplification step (c) are conducted with a pair of primers, the pair of primers comprising a forward primer and a reverse primer, wherein the sequence of the forward primer of the amplification step (b) is identical to the sequence of the forward primer of the co-amplification step (c), and wherein the sequence of the reverse primer of the amplification step (b) is identical to the sequence of the reverse primer of the amplification step (c).

16. The method of claim 13, wherein the PCR is conducted at an annealing temperature of 72° C.

17. The method of claim 13, wherein the PCR is repeated for between about 25 and 45 cycles.

18. The method of claim 17, wherein PCR is repeated for about 35 cycles.

19. The method of claim 1, wherein the genetic locus of interest and the control genetic locus are members of the cytochrome P450 gene family.

20. The method of claim 1, wherein the control genetic locus is a paralogous gene.

21. The method of claim 19, wherein the genetic locus of interest is CYP2D6 and the control genetic locus is CYP2D8.

22. The method of claim 15, wherein one primer of the pair has the sequence set forth in SEQ ID NO. 1 and wherein the other primer of the pair has the sequence set forth in SEQ ID NO. 2.

23. The method of claim 15, wherein one primer of the pair is labeled with an affinity moiety.

24. The method of claim 23, wherein the affinity moiety is biotin.
